(19) United States
(12) Patent Application Publication
(10) Pub. No.: US 2005/0159755 A1
(43) Pub. Date: Jul. 21, 2005

(54) Bone growth via periosteal distraction

(76) Inventor: Ronald B. Odrich, New York, NY (US)

Correspondence Address: NOTARO AND MICHALOS, 100 DUTCH HILL ROAD, SUITE 110, ORANGEBURG, NY, 10962-2100, US

(21) Appl. No.: 10/935,413

(22) Filed: Sep. 7, 2004

Related U.S. Application Data
(63) Continuation-in-part of application No. 10/761,840, filed on Jan. 21, 2004.

(51) Int. Cl. A61F 5/04; A61B 17/56; A61F 2/30; A61F 5/00

(52) U.S. Cl. 606/86; 606/53

(57) ABSTRACT

A periosteal distraction osteogenesis method and apparatus uses a sheet member for covering a surface of living bone that is, in turn, covered by soft periosteal tissue. The sheet member is under the soft tissue and over an area where bone growth outwardly and normally from the bone surface is desired. The method and apparatus requires distracting the sheet member away from the portion of the bone surface at which bone growth is desired, for causing growth of the bone outwardly and normally from the bone surface.

### CROSS REFERENCE TO RELATED APPLICATION

This is a continuation-in-part of application Ser. No. 10/761,840 filed Jan. 21, 2004, which is incorporated here by reference.

### FIELD AND BACKGROUND OF THE INVENTION

The present invention relates generally to the field of osteogenesis (bone growth), and in particular to a new and useful method and apparatus for growing bone in selected areas of a human or animal subject, particularly, but not exclusively, for growing bone at the crest of the jawbone.

It is often desirable to encourage bone to grow. Any bone fracture to be treated, for example, relies on bone growth to knit the bone back together in the fractured area.

The field of distraction osteogenesis is known for causing facial and other bones to grow as exemplified in U.S. Pat. Nos. 5,769,850; 5,810,812; and 5,976,142 to Martin Chin. Also see the article in Vol. 12, No. 2, Spring 200, Orthodontic Dialogue, “Distraction Osteogenesis: A New Frontier in Correcting Dentofacial Deformities.”

In the mouth, when a tooth or teeth are lost, the jawbone in the edentulous area tends to shrink. This poses a particular problem in cases where implants are being considered for replacing the missing tooth or teeth, since a certain minimum amount of bone mass and depth is needed. It has not been possible until now to simply induce a jawbone to grow occlusally (outwardly) from the crest of the bone, or laterally, that is, in a direction that is normal or perpendicular, or crestally or laterally to the outer surface of the bone, without involving complex procedures using grafting materials, membranes, distraction osteogenesis, etc.

There is no teaching in the prior art that is known to the inventor, of any method or apparatus for inducing outward bone growth that is normal, i.e. perpendicular, to the outer bone surface, using magnetic attraction, mechanical biasing or any other means, except for the very different approach of distraction osteogenesis identified above.

### SUMMARY OF THE INVENTION

The periosteal distraction osteogenesis (bone growth) method and apparatus of the present invention comprises applying to a surface of living bone that is covered by soft or periosteal tissue and at which bone growth is desired, a sheet member for covering at least a portion of the bone surface under the soft tissue and distracting the sheet member from the portion of the bone surface at which bone growth is desired for causing growth of bone outwardly and normally from the bone surface.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of a portion of a human jawbone partly covered by soft periosteal tissue including gingival tissue (gum), and with an edentulous or toothless area where the bone has receded, and an apparatus of the present invention;

FIG. 2 is an exploded view of another apparatus of the present invention for pulling a sheet member according to the method of the invention;

FIG. 3 is a sectional view of another apparatus of the present invention;

FIG. 4 is a perspective view of the apparatus of FIG. 3;

FIG. 5 is a sectional view of a still further apparatus of the present invention;

FIG. 6 is a perspective view of the apparatus of FIG. 5;

FIG. 7 is a sectional view of a further apparatus of the present invention; and

FIG. 8 is a perspective view of the apparatus of FIG. 7.

### DESCRIPTION OF THE PREFERRED EMBODIMENTS

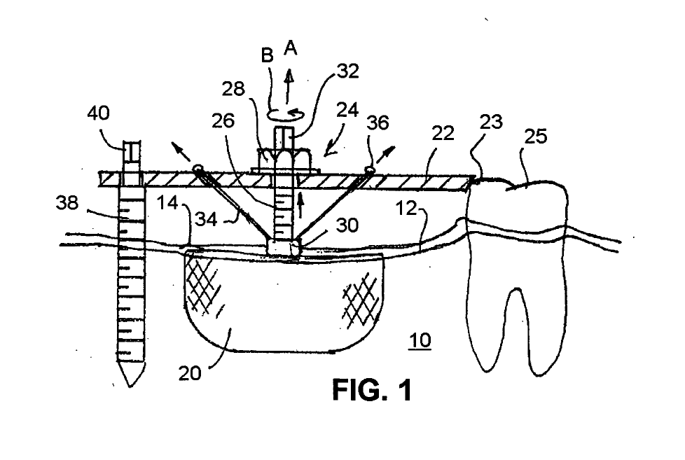

Referring now to the drawings in which like reference numerals are used to refer to the same or functionally similar elements, FIG. 1 illustrates a jawbone 10 with one tooth 25 and having an edentulous or toothless area 12 extending at least to a pin 38. Gingival tissue or gum 14 covers the jawbone and edentulous area and surrounds the tooth in usual fashion. As is know, the jawbone in area 12 tends to shrink or recede after the teeth have been lost. If a dental implant is desired in area 12 to replace the missing teeth, however, a certain minimum amount of bone must be present.

According to the present invention, bone can be induced to grown occlusally, that is outwardly or normally from the crestal surface of the jawbone.

The periosteal distraction osteogenesis method and apparatus of the invention comprises applying to the surface of the living bone 10 that is covered by soft tissue 14 and at which bone growth is desired, a sheet member 20 for covering at least a portion of the bone surface 12 under the soft tissue.

Sheet member 20 can be any grid, screen, membrane of other sheet material, such as, but not limited to the various materials and configurations disclosed in the parent application, as long as the material is bio-compatible in its use over the bone surface and under the soft tissue, and preferably semipermeable to allow fluid (e.g. blood) flow across and through its surface.

Also, although the example of the bone of the jaw is given here, the invention can be used for any bone covered by periosteal tissue as in the parent application, such as but not limited to, long bones of the hands, arms, feet and legs, broad bones such as the front of the chin, vertebrae, pelvic bones, collar bones, skull bones, and other bones of the body, whether human or otherwise.

The invention includes distracting the sheet member 20 away from the portion of the bone surface 12 at which bone growth is desired, for causing growth of bone outwardly and normally from the bone surface. Although magnetic attraction can be used for this purpose as disclosed in the parent application, a more generic mechanism for pulling or pushing the sheet member away from the bone surface, referred to here as “distraction,” can also be used to induce bone growth outwardly (e.g. perpendicularly or normally) of the bone surface.

For this purpose, the invention includes any known or hereafter discovered means for distracting the sheet member 20, such as for mechanically pulling the sheet member away from the bone surface to distract the sheet member.

Alternatively the invention includes means for mechanically pushing the sheet member away from the bone surface to distract the sheet member.

An example of means for pulling the sheet member 20 is shown in FIG. 1 which includes a platform 22 at a fixed location spaced outwardly of the sheet member 20 and a device 24 for mechanically engaging the platform to the sheet member for pulling the sheet member in the direction of arrow A to distract the sheet member from the bone surface.

Device 24 in the example of FIG. 1, comprises a threaded screw 26 threaded into a threaded thimble 28. Screw 26 is mechanically engaged to the platform at thimble 28 and is rotatably connected to the sheet member 20 by a transfer stud 30 which is connected to the sheet member. Using a wrench or like tool engaged to a hex, square or other non-circular projection 32 at the top end of screw 26, and holding thimble 28 against rotation, e.g. using another wrench or any appropriate holding device, screw 26 is turned in the direction of arrow B. For this purpose, thimble 28 may also be non-circular.

This causes screw 26 to ride up in thimble 28 by a small amount to apply upward pulling force to sheet member 20. For the upper jaw the pulling direction is downward but in any case the pulling force is applied away from the bone surface 12 to induce bone growth under the sheet member 20 as a result of the tissue 14 being pulled gently away from the bone.

The outward movement is applied in small increments over a period of weeks to give the bone sufficient time to grow. The amount and scheduling for such pulling depends on the growth rate of bone in the area being treated by the invention, the age and physiological condition of the subject and other factors which the professional using the invention will appreciate.

Another mechanism for pulling the sheet material which can be used in addition to, or instead of the screw-plus-thimble device, is one or more elongated flexible or elastic members 34 for mechanically engaging the platform to the sheet member. Each elongated flexible member 34, may, for example be a length of ortho wire of the type used for orthodontics, which extends through holes in the platform 22 and can be pulled by suitable orthodontic tools and anchored by a bend, bead or knot 36 above platform 22 to maintain tension between the platform and the sheet member. Members 34 may alternatively be elastic bands that exert a continuous but gentle pulling on the sheet 20.

The lower ends of wires 34 can be welded, tied or otherwise fixed to the transfer stud 30 or directly to the sheet member 20.

The elongated flexible members 34 may alternatively comprise elastic bands which can be tightened as needed to pull on sheet member 20.

One side of platform 22 can be supported by the tooth 25 at bevel 23 which can be bonded to the tooth for security. The other side of the platform, or indeed both sides for a completely edentulous area, can be supported by a pin 38 implanted into the bone 10 using currently know implant technology, for example, with a hex or square head 40 for ease of placement by turning the threaded pin 38 into a suitably provided bore in bone 10 using an appropriate tool. The pin may also be tapped into place.

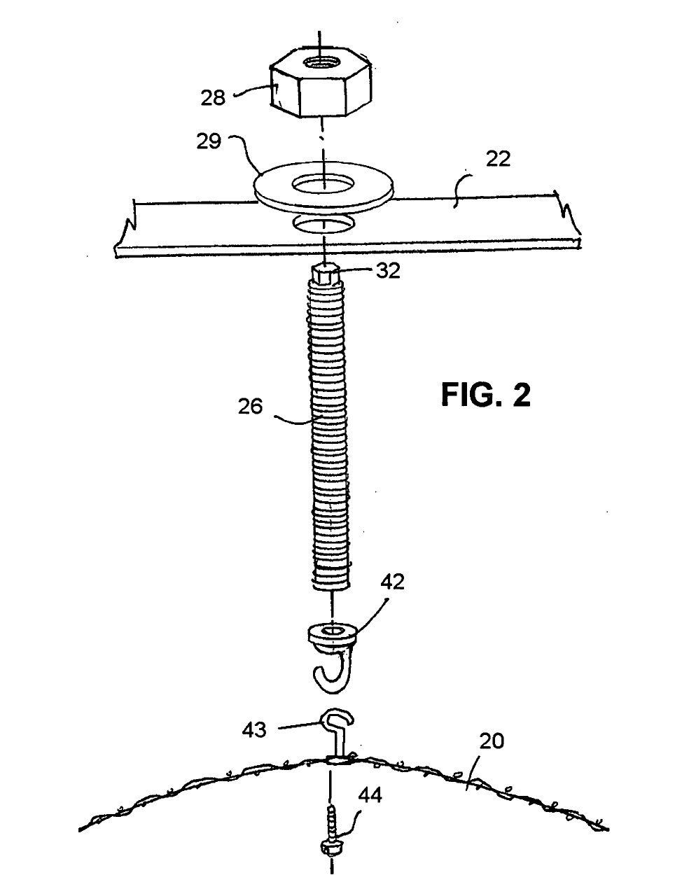

FIG. 2 shows another embodiment of the means for pulling the sheet member 20, this time incorporating the form of a fine wire screen, within or adjacent to a membrane. These means comprise threaded screw 26, that is threadable into thimble or hex nut 28, that is supported on a washer 29 on platform 22. A hook member 42 can be hooked into the mesh of screen 20 and is rotatably connected, e.g. by a small fixing screw 44, to the bottom of screw 26.

Hook 42 can engage an eye 43 that is fixed to or rotatably connected to the sheet 20 and which extends transgingivally (beyond the gum tissue) of the sheet so that the outward pulling force is exerted on the sheet, away from the bone surface.

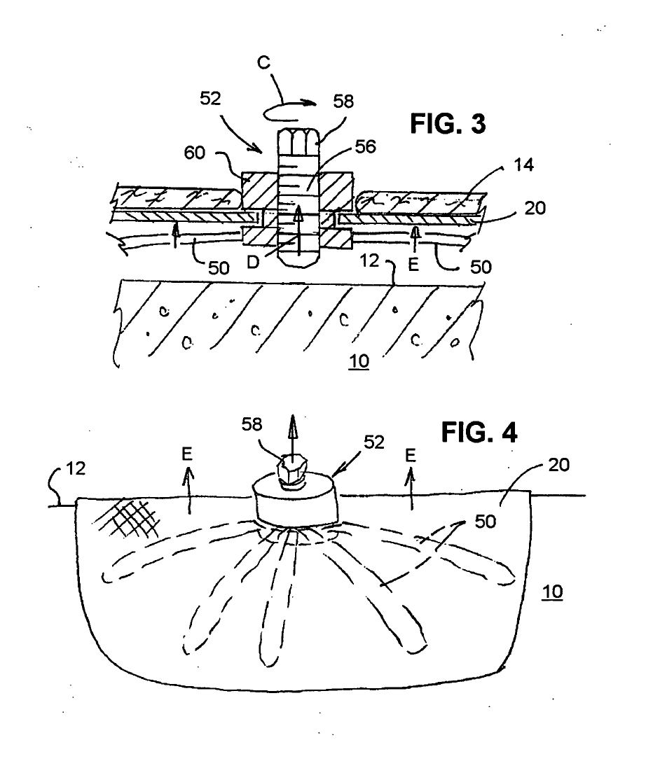

FIGS. 3 to 8 illustrate mechanical distraction means connected to the sheet members for mechanically pulling the sheet members away from the bone surface. Each is used with a fixed and spaced away platform, such as platform 22, which is not shown.

In FIGS. 3 and 4, the mechanical distraction means comprises a plurality of flexible expansion arms 50 extending between the sheet member 20 and the bone surface 12, and jack means 52 connected to the arms for moving at least the inner ends of each arm away from the bone surface 12.

Jack means 52 are connected to the inner end of each arm and include means, e.g. a screw 56, for pulling way from the bone surface 12 to lift each arm away from the bone surface. Screw 56 has a hex or other tool engagement head 58 which is turned in the direction of arrow C in nut 60 that is fixed to sheet member 20. This moves screw 56 in the direction of arrow D, away from the bone surface 12, thus lifting sheet member 20 and the tissue 14 thereon, in the direction of arrows E.

In a more complex mechanism, the inner ends of arms 50 are pivotally connected to nut 60 like the arms of a “molly bolt” to pivot each interior, central arm away from the bone and in that way lift the sheet member and soft tissue via the movement of the peripheral extent of the arms.

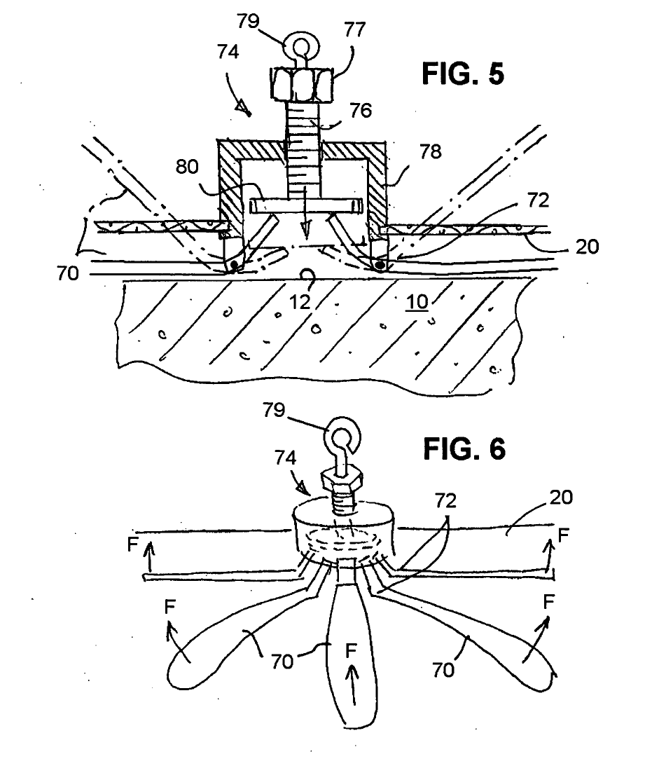

In the embodiment of FIGS. 5 and 6, each arm 70 includes a fulcrum 72 in the form of a bend in each arm, pivotally connected to a housing 78 for pivoting by the action of jack means 74 for pressing against each arm on one side of each fulcrum to raise each arm on an opposite side of each fulcrum in the direction of arrows F. Jack means 74 comprises the housing 78 to which a screw 76 with hex head 77 is threaded. Turning the screw 76 moved a plate or washer 80 connected to the bottom of the screw, downwardly to push down on the inner ends of arms 70. This causes the arms to pivot and their opposite ends to rise to the eventual position shown in phantom line in FIG. 5 to raise the sheet member or grid 20 away from the bone surface. Eyelet 79 holds the entire assembly to the platform 22 for the net outward pulling of the sheet member 20.

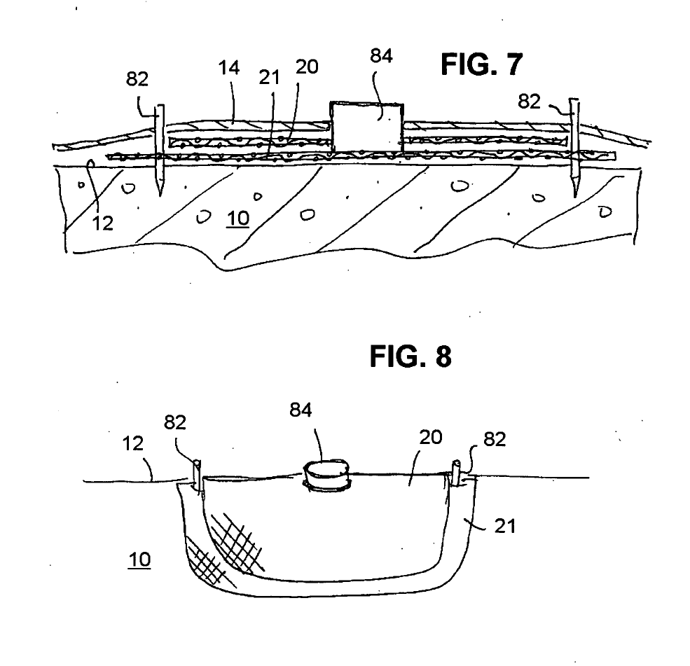

FIGS. 7 and 8 illustrate a further embodiment of the invention there a pair of superimposed sheet members 20 and 21 are held over the bone surface by two or more studs 82 fixed into the bone. The lower or inner sheet member 21 is pierced by the studs while the upper or outer sheet member 20 is held between the studs and is free to move outwardly of the bone surface. A stabilizing base 84 is connected to the lower sheet member 21 and extends outwardly through a hole in the upper sheet member 20. Base 84 may be threaded to cause outer sheet member 20 to rise as base 84 is rotated means for repelling the sheet member 20 and 21 apart can be provided such as by magnetizing the sheet members to have opposite poles or by inducing like electrical charge in each sheet member so that they repel each other. This will tend to raise the outer sheet member 20, inducing bone growth that will eventually encase the inner sheet member 21 but still enlarge the bone crest.

While specific embodiments of the invention have been shown and described in detail to illustrate the application of the principles of the invention, it will be understood that the invention may be embodied otherwise without departing from such principles.

1. A periosteal distraction osteogenesis method comprising:
applying to a surface of living bone that is covered by soft periosteal tissue and at which bone growth is desired, a sheet member for covering at least a portion of the bone surface under the soft tissue; and
distracting the sheet member from the portion of the bone surface at which bone growth is desired for causing growth of bone outwardly and normally from the bone surface.

2. The method of claim 1, including mechanically pulling the sheet member away from the bone surface to distract the sheet member.

3. The method of claim 1, including mechanically pulling the sheet member away from the bone surface to distract the sheet member.

4. The method of claim 1, including providing a platform at a fixed location spaced outwardly of the sheet member and mechanically engaging the platform to the sheet member for pulling the sheet member to distract the sheet member from the bone surface.

5. The method of claim 4, including a threaded screw and thimble for mechanically engaging the platform to the sheet member.

6. The method of claim 4, including at least one elongated member for mechanically engaging the platform to the sheet member.

7. The method of claim 6, wherein the elongated member comprises flexible wire.

8. The method of claim 6, wherein the elongated member comprises an elastic band.

9. The method of claim 4, including at least partly supporting the platform on at least one pin implanted into the bone.

10. The method of claim 4, including at least partly supporting the platform on at least one tooth that is adjacent the bone surface at which bone growth is desired.

11. The method of claim 1, including connecting mechanical distraction means to the sheet member for mechanically pulling the sheet member away from the bone surface.

12. The method of claim 11, wherein the mechanical distraction means comprises a plurality of expansion arms extending between the sheet member and the bone surface, and jack means connected to the arms for moving at least part of each arm away from the bone surface.

13. The method of claim 12, wherein the jack means is connected to each arm and includes means for pulling away from the bone surface to lift each arm away from the bone surface.

14. The method of claim 12, wherein each arm includes a fulcrum, the method including pressing the jack means against each arm on one side of each fulcrum to raise each arm on an opposite side of each fulcrum.

15. The method of claim 1, including providing a second sheet member extending under the first mentioned sheet member, and separating the sheet members from each other to distract the first mentioned sheet member from the bone surface.

16. A periosteal distraction osteogenesis apparatus comprising:
a sheet member for covering at least a portion of a surface of living bone at which bone growth is desired, the sheet member being adapted to extend under soft periosteal tissue covering the bone surface; and
distraction means engaged to the sheet member for distracting the sheet member from the portion of the bone surface at which bone growth is desired for causing growth of bone outwardly and normally from the bone surface.

17. The apparatus of claim 16, wherein the distraction means comprises means connected to the sheet member for mechanically pulling the sheet member away from the bone surface to distract the sheet member.

18. The apparatus of claim 16, wherein the distraction means comprises means connected to the sheet member for mechanically pulling the sheet member away from the bone surface to distract the sheet member.

19. The apparatus of claim 16, including a platform at a fixed location spaced outwardly of the sheet member and means for mechanically engaging the platform to the sheet member for pulling the sheet member to distract the sheet member from the bone surface.

20. The apparatus of claim 19, wherein the means for mechanically engaging comprises a threaded screw and thimble for mechanically engaging the platform to the sheet member.

21. The apparatus of claim 19, including at least one elongated member for mechanically engaging the platform to the sheet member.

22. The apparatus of claim 21, wherein the elongated member comprises flexible wire.

23. The apparatus of claim 21, wherein the elongated member comprises an elastic band.

24. The apparatus of claim 19, including at least one pin adapted to be implanted into the bone for at least partly supporting the platform.

25. The apparatus of claim 19, including means for at least partly supporting the platform on at least one tooth that is adjacent the bone surface at which bone growth is desired.

26. The apparatus of claim 16, wherein the distraction means comprises mechanical distraction means connected to the sheet member for mechanically pulling the sheet member away from the bone surface, the mechanical distraction means comprising a plurality of expansion arms extending between the sheet member and the bone surface, and jack means connected to the arms for moving at least part of each arm away from the bone surface.

27. The apparatus of claim 26, wherein the jack means is connected to each arm and includes means to lift each arm away from the bone surface.

28. The apparatus of claim 26, wherein each arm includes a fulcrum and the jack means pressing against each arm on one side of each fulcrum to raise each arm on an opposite side of each fulcrum.

29. The apparatus of claim 16, including a second sheet member extending under the first mentioned sheet member, and the distraction means comprising means for separating the sheet members from each other to distract the first mentioned sheet member from the bone surface.
